(19) United States
(12) Patent Application Publication
(10) Pub. No.: US 2026/0347753 A1
(43) Pub. Date: Nov. 5, 2026

(54) BIOPROTECTIVE LACTICASEIBACILLUS RHAMNOSUS WITH LOW POSTACIDIFICATION

(71) Applicant: CHR. HANSEN A/S, Hoersholm (DK)

(72) Inventors: Solvej PRECHT, Hoersholm (DK); Mads LUND, Hoersholm (DK); Helle Skov GULDAGER, Hoersholm (DK); Vera Kuzina POULSEN, Hoersholm (DK); Jeorgos TRIHAAS, Hoersholm (DK); Lise Soendegaard KRISTENSEN, Hoersholm (DK); Anisha GOEL, Hoersholm (DK); Sophia HARRAND, Hoersholm (DK); Louise Essendrup STEFFENSEN, Hoersholm (DK)

(73) Assignee: CHR. HANSEN A/S, Hoersholm (DK)

(21) Appl. No.: 19/140,036

(22) PCT Filed: Dec. 21, 2023
(86) PCT No.: PCT/EP2023/087298

§ 371 Date: Jun. 17, 2025

(30) Foreign Application Priority Data
Dec. 27, 2022 (EP) 22216795.9

(51) Int. Cl. C12N 1/20; A23C 9/123; A23C 13/16; A61K 35/00; A61K 35/747

(52) U.S. Cl. CPC C12N 1/20; A23C 9/1234; A23C 13/165; A61K 35/747; A23V 2400/175; A61K 2035/115

(57) ABSTRACT

The present application is in the field of dairy technology. It relates to the bacterium Lacticaseibacillus rhamnosus DSM 34195 or its mutants, as well as uses thereof for producing food, feed or pharmaceutical products, in particular fermented milk products. Lacticaseibacillus rhamnosus bacteria DSM 34195 has low post-acidification in the products and provides antifungal effects. The invention also provides compositions comprising Lacticaseibacillus rhamnosus DSM 34195 or mutants thereof.

### FIELD OF THE INVENTION

The present application relates to Lacticaseibacillus rhamnosus bacteria with low post-acidification in fermented milk products and having antifungal effects.

### BACKGROUND OF THE INVENTION

Lactic acid bacteria (LAB) have been used over decades for increasing the shelf life of food products. During fermentation, lactic acid and other organic compounds are produced by the lactic acid bacteria, thereby reducing the pH of the food product and consequently making it unfavorable to the growth of undesired microorganisms, such as yeast and mold.

Bioprotection is defined as the extension of shelf life and enhanced safety of foods using natural or controlled antimicrobial compounds. In dairy products, spoilage by mold and yeast cells is one of the major problems negatively affecting shelf life. In the past decade, considerable efforts have been invested to explore the bioprotective potential of LAB, to identify new strains with bioprotective properties from various food sources, as well as to elucidate the mechanisms behind the observed antifungal activity. Numerous metabolites produced by LAB have been identified as having antifungal activities.

Further studies have identified that competitive exclusion of a limited resource by different organisms is a major mechanism of fungal growth inhibition by Lactic acid bacteria. In particular, the depletion of the essential trace element manganese is a major bioprotective mechanism of lactic acid bacteria in dairy products. It was also shown that manganese scavenging is an active mechanism and that it requires energy to maintain a high manganese gradient (Siedler et al. “Competitive exclusion is a major bioprotective mechanism of lactobacilli against fungal spoilage in fermented milk products.” Applied and environmental microbiology 86.7 (2020)).

At the same time, it has been found that high antifungal activity of bioprotective strains is generally accompanied by high activity, which causes post-acidification, i.e. continuation of acidification after termination of fermentation. The production of bioprotective compounds in LAB usually shows growth-associated kinetics and is therefore expected to cease if the growth is reduced (Lv et al. “Modelling the production of nisin by Lactococcus lactis in fed-batch culture.” Applied microbiology and biotechnology 68.3 (2005): 322-326). Since milk acidification is typically associated with growth (Dandoy et al. “The fast milk acidifying phenotype of Streptococcus thermophilus can be acquired by natural transformation of the genomic island encoding the cell-envelope proteinase PrtS.” Microbial cell factories. Vol. 10. No. S1. BioMed Central, 2011), it is expected that strains exhibiting reduced post-acidification will also have reduced bioprotective effects.

WO2021239574 discloses Lactobacillus rhamnosus DSM 33515, a bioprotective strain that was described to exhibit a combination of reduced post-acidification and high bioprotective effects.

For economic reasons, there is a constant need for improved solutions which are effective for controlling microbial spoilage or contamination while providing reduced post-acidification.

### SUMMARY OF THE INVENTION

The present application therefore provides a bacterium of the species Lacticaseibacillus rhamnosus deposited as DSM 34195 or a mutant Lacticaseibacillus rhamnosus of the deposited bacteria. The mutant may exhibit substantially the same or improved bioprotective effect against yeast and mold as well as reduced post-acidification compared to DSM 34195. The mutant is obtained by using as starting material DSM 34195. The mutant is different from DSM 33515 and DSM 23035. The mutant may have retained or improved properties compared to DSM 34195, and may have improved properties compared to DSM 33515. The properties discussed in this paragraph and in the present disclosure are antifungal effect or activity (in other words: bioprotective effect against yeast and mold) and reduced post-acidification.

The invention additionally provides a composition comprising bacteria of the species Lacticaseibacillus rhamnosus as described above. In one embodiment, the composition further comprises cryoprotectants, lyoprotectants, antioxidants, nutrients, fillers, flavorants or mixtures thereof. In one embodiment, the bacteria are in a concentration of at least 10⁹ colony forming units (CFU)/g, or in a concentration of at least 10¹⁰ CFU/g, or in a concentration of at least 10¹¹ CFU/g in the composition. In a preferred embodiment, the composition is frozen or freeze-dried. In one embodiment, the composition further comprises a starter culture.

The invention further provides a method of producing a fermented milk product comprising adding bacteria of the species Lacticaseibacillus rhamnosus strain as described above or a composition comprising the same to milk or to a milk product and fermenting the mixture at a temperature between about 22 °C and about 43 °C, such as between 22 °C and 43 °C, until a pH of 4.6 or less than 4.6 is reached.

The invention provides a fermented milk product comprising bacteria of the species Lacticaseibacillus rhamnosus as described above. Preferably, the fermented milk product is obtained by the method as mentioned above. In a further embodiment, the fermented milk product maintains a pH above 3.8 when stored for at least 28 days at 25 °C. In another embodiment, the bacteria of the species Lacticaseibacillus rhamnosus are present in a concentration of at least 10⁷ CFU/g.

In addition, the invention provides food, feed or pharmaceutical product comprising the bacteria of the species Lacticaseibacillus rhamnosus as described above or a composition comprising the same. In a preferred embodiment, the food, feed or pharmaceutical product is obtained by the method as mentioned above.

### BRIEF DESCRIPTION OF THE FIGURES

FIG. 1 shows post acidification of L. rhamnosus DSM 34195 and DSM 33515 at four different final inoculation dosages in 96 low-well plate assay.

FIG. 2 shows mold inhibition of L. rhamnosus DSM 34195 and DSM 33515 at four different final inoculation dosages in 96 low-well plate assay.

FIG. 3 shows yeast inhibition of L. rhamnosus DSM 34195 and DSM 33515 at four different final inoculation dosages in 96 low-well plate assay.

FIG. 4 shows the growth of yeasts prepared from milk fermented with a starter culture only (Reference), or with a starter culture in combination with L. rhamnosus bacteria DSM 33515 or with a starter culture in combination with L. rhamnosus DSM 34195 (3 different dosages).

FIG. 5 shows the growth of six different molds added as contaminant to agar plates made from milk fermented with starter culture only (Reference), or starter culture in combination with L. rhamnosus bacteria DSM 33515 or a starter culture in combination with L. rhamnosus DSM 34195 (3 different dosages).

FIG. 6 shows the growth of three different molds added as contaminant to agar plates made from milk (with or without 10% sucrose) fermented with starter culture only (YF-812 or Advance 2.0) (Reference), or starter culture in combination with L. rhamnosus bacteria DSM 33515 or starter culture in combination with L. rhamnosus DSM 34195 (3 different dosages).

FIG. 7 shows the growth of yeasts prepared from milk (with 10% sucrose) fermented with the starter culture only (Reference), or with a starter culture in combination with L. rhamnosus bacteria DSM 33515 or with a starter culture in combination with L. rhamnosus DSM 34195 (3 different dosages). FIGS. 7A and 7B show the results using the starter culture YF-L812 and Advance 2.0, respectively.

FIG. 8 shows the growth of yeasts prepared from milk (without sucrose) fermented with the starter culture only (Reference), or with a starter culture in combination with L. rhamnosus bacteria DSM 33515 or with a starter culture in combination with L. rhamnosus DSM 34195 (3 different dosages). FIGS. 8A and 8B show the results using the starter culture YF-L812 and Advance 2.0, respectively.

FIG. 9 shows the growth of three different molds added as contaminant to agar plates made from milk fermented with starter culture only (YoFlex Mild 1.0 or YoFlex Premium 1.0) (Reference), or starter culture in combination with L. rhamnosus bacteria DSM 33515 or a starter culture in combination with L. rhamnosus DSM 34195 (3 different dosages).

FIG. 10 shows the growth of yeast prepared from milk fermented with the starter culture only F-DVS YoFlex Mild 1.0 or YoFlex Premium 1.0) (Reference), or with a starter culture in combination with L. rhamnosus bacteria DSM 33515 or with a starter culture in combination with L. rhamnosus DSM 34195 (3 different dosages). FIGS. 10A and 10B show the growth of Debaryomyces (D.) hansenii and Torulaspora delbrueckii, respectively.

FIG. 11 shows the pH development in fermented milk products over time when stored at 25±1 °C for 28 days. The products were fermented with starter culture only (Reference), or with starter culture in combination with DSM 33515 or with starter culture in combination with DSM 34195. FIGS. 11A and 11B show the results using the starter culture YF-L812 and Advance 2.0, respectively.

FIG. 12-14 shows the growth of six different molds added as contaminant to agar plates made from crema ácida without L. rhamnosus (Reference) or with L. rhamnosus bacteria DSM 33515 or L. rhamnosus DSM 34195 and incubated at different temperatures.

FIG. 15 shows the growth of D. hansenii yeast in crema ácida prepared without L. rhamnosus (Reference) or with L. rhamnosus bacteria DSM 33515 or L. rhamnosus DSM 34195.

FIG. 16-18 shows the pH development in crema ácida over time when stored at at 7 °C, 12 °C and 25 °C for 28 days. The products were without L. rhamnosus (Reference) or with L. rhamnosus bacteria DSM 33515 or L. rhamnosus DSM 34195.

### DETAILED DESCRIPTION OF THE INVENTION

Food cultures with bioprotective effects offering a safe add on solution for traditionally fermented products are available. These bioprotective cultures are used in combination with normal starter cultures to co-ferment milk to a fermented product. During the fermentation, it will exert bioprotective effects and thus provide an extended shelf-life of the fermented products against molds and yeasts. Fermentation of many dairy products, such as yoghurt, are stopped and the product is cooled down at a specific pH, after fermentation the bacteria are frequently still active during storage. Further lactate is produced and the process is known as post-acidification. The resulting lower pH of the final product has a negative sensory impact on the product and is therefore undesirable. There is a need to develop new and improved bioprotective cultures that exhibit a combination of reduced post-acidification and high bioprotective effects. For example, the strain Lactobacillus rhamnosus DSM 33515 was previously disclosed as a bioprotective strain which exhibits a combination of reduced post-acidification and high bioprotective effects. The present application now provides improved a bioprotective strain DSM 34195 and mutants thereof that offer further advantages over DSM 33515, as demonstrated in the Examples.

In the present context, the term “mutant” should be understood as a strain derived from a strain of the invention, for example by means of e.g. genetic engineering, radiation and/or chemical treatment. It is preferred that the mutant is a functionally equivalent mutant, e.g. a mutant that has substantially the same, or improved, properties in particular in relation to the effects on reducing post-acidification and/or bioprotection, 25 as the deposited strain. Respective mutants represent embodiments of the present application. The term “mutant” in particular refers to a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant. A mutant may have been subjected to several mutagenization treatments (a single treatment should be understood one mutagenization step followed by a screening/selection step), but it is presently preferred that no more than 20, or no more than 10, or no more than 5, treatments (or screening/selection steps) are carried out. In a presently preferred mutant, less than 5%, or less than 1% or even less than 0.1% of the nucleotides in the bacterial genome have been shifted with another nucleotide, or deleted, compared to the mother strain.

Extensive screening of 10,000 mutants of DSM 23035 led to the identification of strain DSM 34195. This strain can be used at lower inoculation dosage while achieving low post-acidification and high antifungal activity. Furthermore, the strain has been shown to be combinable with different starter cultures and milk bases, showing better compatibility than DSM 33515. Furthermore, DSM 34195 exhibits good sensorial properties, with its clean and mild taste and lack of off-flavor notes.

The present application thus provides the Lacticaseibacillus rhamnosus strain DSM 34195 and mutants maintaining the advantageous properties of DSM34195, when compared under the same conditions.

For post-acidification, the comparison can be carried out for example as described below:
preparing a homogenized milk base consisting of 2.8% protein, 1.2% fat and 10% sucrose, heat-treating the milk base at 95±1 °C for 5 min and cooling immediately,
inoculating the milk base with the starter culture YF-L812 at 500 U per 2500 L,
inoculated the milk base with L. rhamnosus DSM 34195 and, in parallel, a mutant to be compared, in total concentration of 1×10⁷ CFU/g, fermenting at 43 °C until pH of 4.60+0.1 is reached and cooling to 25±1 °C, and placing the samples at cold storage at 5-7 °C, and
storing the samples at 25 #1 °C for 28 days and measuring pH on day 1, 7, 14, 21 and 28.

For antifungal effect, the comparison can be carried out for example as described below:
preparing a homogenized milk base consisting of 2.8% protein, 1.2% fat and 10% sucrose, heat-treating the milk base at 95±1 °C for 5 min and cooling immediately,
inoculating the milk base with the starter culture YF-L812 at 500 U per 2500 L,
inoculated the milk base with L. rhamnosus DSM 34195 and, in parallel, a mutant to be compared, in total concentration of 1×10⁷ CFU/g,
fermenting at 43 °C until pH of 4.60+0.1 was reached and cooling in a cooling chamber,
inoculating the fermented milk sample with 50 cfu/g D. hansenii CHCC16374, and
I storing the samples at 7 °C for 28 days and measuring the total yeast count during storage on day 1, 7, 14, 21 and 28.

The Lacticaseibacillus rhamnosus strains of present application have particular advantages as they reduce the risk of post-acidification while exhibiting antifungal activity, thus improves the storage stability of food products made with these bacteria, in particular the storage stability under conditions above refrigeration temperatures.

In the context of present application, a “mold” is a fungus that grows in the form of multi-cellular filaments called hyphae. The term “inhibit” in relation to molds refers to a decrease in the growth or sporulation or a reduction in the number or in the concentration of molds, for example in food products and/or on the surface of food products comprising the bacteria of the present application in relation to food products which do not comprise such bacteria. The extent of inhibition provided by the Lacticaseibacillus rhamnosus strain of present application is preferably determined by growth on agar solidified fermented milk in the presence and absence of Lacticaseibacillus rhamnosus bacteria. Examples of molds are members of the genus Penicillium, such as Penicillium solitum, Penicillium brevicompactum, Penicillium crustosum, Penicillium roqueforti, Penicillium paneum and Penicillium carneum.

Yeasts are fungi growing as single cells. The Lacticaseibacillus rhamnosus strain of the present application, i.e., the strain deposited as DSM 34195 and mutants maintaining the advantageous properties inhibit growth of molds and can further inhibit growth of yeasts. In relation to the growth of yeasts the term “inhibit” also refers to a decrease in the growth or a reduction in the number or in the concentration of yeasts, for example in food products and/or on the surface of food products comprising the bacteria of the present application in relation to food products which do not comprise such bacteria. Again, the extent of inhibition provided by the Lacticaseibacillus rhamnosus strain of present application is preferably determined by growth on agar solidified fermented milk in the presence and absence of Lacticaseibacillus rhamnosus bacteria. Examples of yeasts are members of the genus Debaryomyces and Torulaspora, such as Debaryomyces hansenii and Torulaspora delbrueckii.

The use of the terms “a” and “an” and “the” and similar referents in the context of describing the invention (especially in the context of the following claims) are to be constructed to cover both the singular and the plural, unless otherwise indicated herein or clearly contradicted by context.

Respective compositions may comprise numerous further bacteria including LABs. The term “lactic acid bacteria” or “LAB” is used to refer to food-grade bacteria producing lactic acid as the major metabolic end-product of carbohydrate fermentation. These bacteria are related by their common metabolic and physiological characteristics and are usually Gram-positive, low-GC, acid tolerant, non-sporulating, non-respiring, rod-shaped bacilli or cocci. During the fermentation stage, the consumption of lactose by these bacteria causes the formation of lactic acid, reducing the pH and leading to the formation of a protein coagulum. These bacteria are thus responsible for the acidification of milk and for the texture of dairy product. As used herein, the term “lactic acid bacteria” encompasses, but is not limited to, bacteria belonging to the genus of Lactobacillus spp., Bifidobacterium spp., Streptococcus spp., Lactococcus spp., such as Lactobacillus delbrueckii subsp. bulgaricus, Streptococcus thermophilus, Lactobacillus lactis, Bifidobacterium animalis, Lactococcus lactis, Lactobacillus paracasei, Lactobacillus plantarum, Lactobacillus helveticus, Lactobacillus acidophilus, Bifidobacterium breve and Leuconostoc spp.

A preferred composition of the present application is therefore characterized in that the composition further comprises at least one further bacterium selected from one or more of the following genera and species Lactobacillus spp., Bifidobacterium spp., Streptococcus spp., Lactococcus spp., such as Lactobacillus delbrueckii subsp. bulgaricus, Streptococcus thermophilus, Lactobacillus lactis, Bifidobacterium animalis, Lactococcus lactis, Lactobacillus paracasei, Lactobacillus plantarum, Lactobacillus helveticus, Lactobacillus acidophilus, Bifidobacterium breve and Leuconostoc spp.

In a particularly preferred embodiment, the compositions of the present application comprise bacteria of the species Lacticaseibacillus rhamnosus deposited as DSM 34195 or a mutant Lacticaseibacillus rhamnosus obtainable from the deposited bacteria and one or more further bacteria. In one embodiment, several different strains of the Lacticaseibacillus rhamnosus bacteria are combined.

The composition of the present application may additionally comprise cryoprotectants, lyoprotectants, antioxidants, nutrients, fillers, flavorants or mixtures thereof. The composition may be in frozen or freeze-dried form. The composition preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as cryoprotectants and lyoprotectants are known to a skilled person in the art. Suitable cryoprotectants or lyoprotectants include mono-, di-, tri- and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate). Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorants.

LAB are most commonly added to milk in the form of a starter culture. The term “starter” or “starter culture” as used in the present context refers to a culture of one or more food-grade microorganisms, in particular to lactic acid bacteria, which are responsible for the acidification of the milk base. Starter cultures may be fresh but are most frequently frozen or freeze-dried. These products are also known as “Direct Vat Set” (DVS) cultures and are produced for direct inoculation of a fermentation vessel or vat for the production of a dairy product, such as a fermented milk product or a cheese. Respective starter cultures are commercially available from numerous sources, including Premium 1.0, YF-L812, Mild 1.0, Advance 2.0, which are commercially available from Chr. Hansen containing mixtures of Streptococcus thermophilus and Lactobacillus delbrueckii subsp. bulgaricus. Preferably, the starter culture comprises Streptococcus thermophilus and Lactobacillus delbrueckii subsp. bulgaricus.

In one aspect the present application therefore provides compositions in the form of a solid frozen or freeze-dried starter culture comprising lactic acid bacteria in a concentration of at least 10⁹ colony forming units (CFU)/g, or in a concentration of at least 10¹⁰ CFU/g, or in a concentration of at least 10¹¹ CFU/g. These starter cultures further comprise bacteria of the species Lacticaseibacillus rhamnosus deposited as DSM 34195 or a mutant Lacticaseibacillus rhamnosus obtainable from the deposited bacteria.

In the context of the present application, the term “milk” is broadly used in its common meaning to refer to liquids produced by the mammary glands of animals or by plants. In accordance with the present application the milk may have been processed and the term “milk” includes whole milk, skim milk, fat-free milk, low fat milk, full fat milk, lactose-reduced milk, or concentrated milk. Fat-free milk is non-fat or skim milk product. Low-fat milk is typically defined as milk that contains from about 1% to about 2% fat. Full fat milk often contains 2% fat or more. The term “milk” is intended to encompass milks from different mammal and plant sources. Mammal sources of milk include, but are not limited to cow, sheep, goat, buffalo, camel, lama, mare and deer. Plant sources of milk include, but are not limited to, milk extracted from soybean, pea, peanut, barley, rice, oat, quinoa, almond, cashew, coconut, hazelnut, hemp, sesame seed and sunflower seed. In the methods and products of the present application, milk derived from cows is most preferably used as a starting material for the fermentation.

The term “milk” also includes fat-reduced and/or lactose-reduced milk products. Respective products can be prepared using methods well known in the art and are commercially available. Lactose-reduced milk can be produced according to any method known in the art, including hydrolyzing the lactose by lactase enzyme to glucose and galactose, or by nanofiltration, electrodialysis, ion exchange chromatograph and centrifugation.

The term “milk product” or “milk base” is broadly used in the present application to refer to a composition based on milk or milk components which can be used as a medium for growth and fermentation of LAB. The milk product or base comprises components derived from milk and any other component that can be used for the purpose of growing or fermenting LAB.

Prior to fermentation, the milk substrate may be homogenized and pasteurized according to methods known in the art. “Homogenizing” as used herein means intensive mixing to obtain a soluble suspension or emulsion. If homogenization is performed prior to fermentation, it may be performed so as to break up the milk fat into smaller sizes so that it no longer separates from the milk. This may be accomplished by forcing the milk at high pressure through small orifices. “Pasteurizing” as used herein means treatment of the milk substrate to reduce or eliminate the presence of live organisms, such as microorganisms. Preferably, pasteurization is attained by maintaining a specified temperature for a specified period of time. The specified temperature is usually attained by heating. The temperature and duration may be selected in order to kill or inactivate certain bacteria, such as harmful bacteria. A rapid cooling step may follow.

The present application further provides methods of, wherein the fermented product is stored at a temperature above 7 °C, preferably at a temperature between 7 °C and 25 °C. The product may be stored at any time but is preferably stored for a period of at least 14 days and wherein the pH of the fermented milk product is maintained above pH 4.0 during storage.

The invention further provides methods of producing a food, feed or pharmaceutical product comprising a method of producing a fermented milk product as described above and the food, feed or pharmaceutical product obtainable by this method.

Fermentation is carried out to produce food products, feed products or pharmaceuticals. The terms “fermented milk product”, “food” or “feed” product refer to products obtainable by the fermentation methods of the present application and include cheese, yoghurt, fruit yoghurt, yoghurt beverage, strained yoghurt (Greek yoghurt, Labneh), quark, fromage frais and cream cheese. The term food further encompasses other fermented food products, including fermented meat, such as fermented sausages, and fermented fish products. Examples of fermented milk products include crema ácida and sour cream.

The term “cheese” is understood to encompass any cheese, including hard, semi-hard and soft cheeses. Examples of cheeses include cottage cheese, tvorog, quarg, feta, cheddar, parmesan, mozzarella, emmentaler, danbo, gouda, edam, feta-type like UF-feta, soft cheese, pasta filata, continental cheese, blue cheeses, brine cheeses like white brined cheese, queso fresco Camembert and Brie. The person skilled in the art knows how to convert the coagulum into cheese, methods can be found in the literature, see e.g., Kosikowski, F. V., and V. V. Mistry, “Cheese and Fermented Milk Foods”, 1997, 3rd Ed. F. V. Kosikowski, L. L. C. Westport, CT. As used herein, a cheese which has a NaCl concentration below 1.7% (w/w) is referred to as a “low-salt cheese”.

In the context of the present application, the term “yoghurt” refers to products comprising Streptococcus thermophilus and Lactobacillus delbrueckii subsp. bulgaricus and optionally other microorganisms such as Lactobacillus delbrueckii subsp. lactis, Bifidobacterium animalis subsp. lactis, Lactococcus lactis, Lactobacillus acidophilus and Lactobacillus paracasei, or any microorganism derived therefrom. The lactic acid strains other than Streptococcus thermophilus and Lactobacillus delbrueckii subsp. bulgaricus, are included to give the finished product various properties, such as the property of promoting the equilibrium of the flora. As used herein, the term “yoghurt” encompasses set yoghurt, stirred yoghurt, drinking yoghurt, Petit Suisse, heat treated yoghurt, strained or Greek style yoghurt characterized by a high protein level and yoghurt-like products.

In particular, term “yoghurt” encompasses, but is not limited to, yoghurt as defined according to French and European regulations, e.g. coagulated dairy products obtained by lactic acid fermentation by means of specific thermophilic lactic acid bacteria only (i.e. Lactobacillus delbrueckii subsp. bulgaricus and Streptococcus thermophilus) which are cultured simultaneously and are found to be live in the final product in an amount of at least 10 million CFU (colony-forming unit)/g. Yoghurts may optionally contain added dairy raw materials (e.g. cream) or other ingredients such as sugar or sweetening agents, one or more flavoring(s), fruit, cereals, or nutritional substances, especially vitamins, minerals and fibers, as well as stabilizers and thickeners. Optionally the yoghurt meets the specifications for fermented milks and yoghurts of the AFNOR NF 04-600 standard and/or the codex StanA-IIa-1975 standard. In order to satisfy the AFNOR NF 04-600 standard, the product must not have been heated after fermentation and the dairy raw materials must represent a minimum of 70% (m/m) of the finished product.

### Taxonomy

Lactobacillus rhamnosus is now known as Lacticaseibacillus rhamnosus as described in Zheng et al., Int. J. Syst. Evol. Microbiol. DOI 10.1099/ijsem.0.004107.

### Deposits and Expert Solution

The applicant requests that a sample of the deposited microorganisms stated below may only be made available to an expert, subject to available provisions governed by Industrial Property Offices of States Party to the Budapest Treaty, until the date on which the patent is granted.

TABLE 1
The applicant has made the following deposits at a Depositary institution
having acquired the status of international depositary authority
under the Budapest Treaty on the International Recognition of the
Deposit of Microorganisms for the Purposes of Patent Procedure:
Leibniz Institute DSMZ-German Collection of Microorganisms and
Cell Cultures Inhoffenstr. 7B, 38124 Braunschweig, Germany.

| Strain | Accession No. | Deposit date |
|---|---|---|
| Lacticaseibacillus rhamnosus | DSM 23035 | 2012 Feb. 2 |
| Penicillium solitum | DSM 32093 | 2015 Jul. 16 |
| Penicillium brevicompactum | DSM 32094 | 2015 Jul. 16 |
| Lacticaseibacillus rhamnosus | DSM 33515 | 2020 May 5 |
| Penicillium crustosum | DSM 33517 | 2020 May 5 |
| Penicillium roqueforti | DSM 33518 | 2020 May 5 |
| Penicillium paneum | DSM 33519 | 2020 May 5 |
| Penicillium carneum | DSM 33520 | 2020 May 5 |
| Lacticaseibacillus rhamnosus | DSM 34195 | 2022 Mar. 1 |

### Examples

### Example 1 Generation of L. rhamnosus DSM 34195

### 1.1 Mutant Pool

Ethyl methanesulfonate (EMS) mutagenesis was used to obtain a mutant pool from the mother strain L. rhamnosus DSM 23035. Pre-experiments were carried to establish efficacy and killing rate of EMS for this strain. A killing rate of ≥95% was targeted. Based on this, 1 ml overnight culture (OD₆₂₀=3-4) in MRS-Difco was added with 15 μl EMS. The cultures were then incubated for 4 hours at 37 °C and then diluted into series ranging from 10-2 to 10-6. Subsequently, the diluted cultures are spread on MRS-Difco agar to test for cell count.

### 1.2 Selection of DSM 34195

Mutant strains were selected from the mutant pool by high-throughput screening, which included mutant hit picking from agar, growth of mutants in broth, milk acidification and post-acidification, selection of mutants with low post-acidification antifungal assessment.

In more detail, MRS-Difco agar pH 6.5 was poured into low profile square bioassay dish (Corning 431301). The mutant pool was spread on agar using sterile glass beads and incubated for 2 days at 37 °C anaerobically. Approximately 10,000 colonies were hit-picked using QPix2 colony picker (Genetix) into 200-μl 96-well micro-titer plates containing MRS-Difco broth. After an overnight incubation of the micro-titer plates under anaerobic conditions at 37 °C, 1% of the overnight inoculum was used to inoculate milk in 2-ml 96-well micro-titer plates. A homogenized milk base consisting of 2.8% protein, 1.2% fat and 10% sucrose was heat-treated at 95±1 °C for 5 min and cooled immediately was used. Color-of-pH method was used to monitor milk acidification, as described in Poulsen et al. “High-throughput screening for texturing Lactococcus strains.” FEMS microbiology letters 366.2 (2019): fnz001. The mutants were incubated in milk added with the starter culture YF-L812 and a pH indicator at 40 °C for 5-6 h until pH 4.55 was reached in control wells containing wild-type strains. YF-L812 is a commercial culture from Chr. Hansen A/S that contains Lactobacillus delbrueckii subsp. bulgaricus and Streptococcus thermophilus.

After identification of the mutants with the lowest post-acidification based on the milk acidification data, the selected mutant strains were collected in 200-μl 96-well micro-titer plates and tested for their antifungal activity. For the precultures, the mutant strains have been grown in BD5-1 media overnight at 37 °C. Afterwards, 10 μl of the preculture was used to inoculate into 2 ml B-milk (ISO 26323:2009) supplemented with 2% sucrose and the starter culture YF-812 in 96 micro-titer plates. The milk was incubated at 40 °C for approx. 6 hours until a pH of 4.55 was reached in control wells containing wild-type strains. The micro-titer plates were stored at 4 °C overnight. Then, 150 μl of the fermented milk was transferred into 200-μl 96-well micro-titer plates and inoculated with approx. 40 Debaryomyces hansenii CHCC16374 (Chr. Hansen Culture Collection) cells/ml. The 200-μl micro-titer plates were stored at 17 °C for 5 days and afterwards a serial dilution row was spotted on YGC-plates for yeast enumeration based on a scale from 0 (no yeast growth) to 5 (confluent growth). The 2-ml micro-titer plates with the remaining fermented milk sample were stored at room temperature for 14 days to measure post-acidification. Mutant strain L. rhamnosus DSM 34195 was selected as a result.

### Example 2 Antifungal Effect and Post-Acidification During Storage in 96 Low-Well Plate Assay

2.1 Post-Acidification of L. rhamnosus DSM 34195 and L. rhamnosus DSM 33515

A homogenized milk base consisting of 2.8% protein, 1.2% fat and 10% sucrose was heat-treated at 95±1 °C for 5 min and cooled immediately. To follow the acidification, a pH indicator was added to the milk and the color change was measured as previously described in section 1.2 but the color values were not converted to pH.

A commercial starter culture containing Streptococcus thermophilus and Lactobacillus delbrueckii subsp. bulgaricus (F-DVS YF-L812) was used to inoculate at 0.02% (v/w) and 270 μL was pipetted into each well. Individual wells were then inoculated with 30 μL of L. rhamnosus DSM 34195 or DSM 33515 to four different final inoculation dosages: 5×10⁶ CFU/g, 1×10⁷ CFU/g, 2×10⁷ CFU/g and 3×10⁷ CFU/g. This was done in 12 replicates across multiple 96 low-well plates. Reference wells were also prepared only containing starter culture (“Blank”). The 96 low-well plates were incubated at 43±1 °C until an equivalent of pH 4.55+0.5 was reached. pH was monitored using the added pH indicator as a pH equivalent. The plates were scanned from the bottom and color values were monitored until a color value equivalent of 4.55 was reached. Separate plates were made for monitoring post-acidification and antifungal effect.

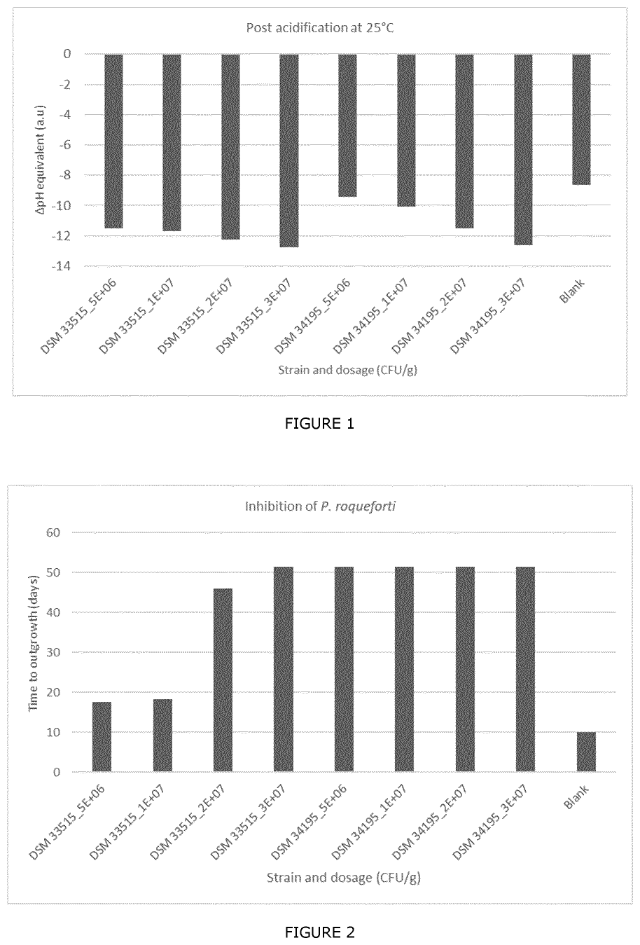

Post-acidification was monitored at 25±1 °C by incubating plates on top of scanners and scanning continuously every 2 hours for 21 days. The change in color value (ΔpH equivalent) during the incubation was then used to indicate post-acidification and shown in FIG. 1.

The results presented in FIG. 1 show that increasing dosages of L. rhamnosus DSM 34195 and DSM 33515 resulted in more post-acidification. In particular, when comparing the same inoculation dosage, L. rhamnosus DSM 34195 shows reduced post-acidification compared to DSM 33515 at all examined dosages.

2.2 Antifungal Effect of L. rhamnosus DSM 34195 and L. rhamnosus DSM 33515 Against Penicillium roqueforti

Spore suspension of P. roqueforti DSM 33518 was spotted on the surface of the fermented yogurt in the 96 low-well plates in concentration of 50 spores per well. The plates were then incubated at 7±1 °C, with a scanner scanning the surface of the plates, for 52 days. The resulting color changes on the surface was used to indicate P. roqueforti inhibition. Time to outgrowth is estimated from the color values and shows the inhibition of P. roqueforti.

The results presented in FIG. 2 show that L. rhamnosus DSM 34195 inhibits P. roqueforti at all examined dosages, whereas L. rhamnosus DSM 33515 shows low inhibition at 5×10⁶ CFU/g and 1×10⁷ CFU/g, almost full inhibition at 2×10⁷ CFU/g and full inhibition at 3×10⁷ CFU/g.

2.3 Antifungal Effect of L. rhamnosus DSM 34195 and L. rhamnosus DSM 33515 Against Debaryomyces hansenii

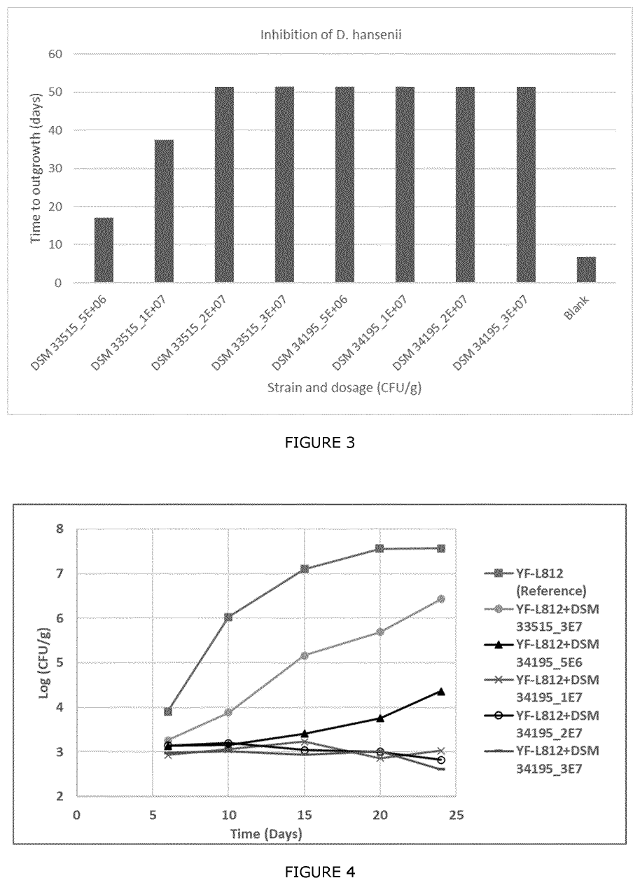

Cell suspension of D. hansenii CHCC16374 was spotted on the surface of the fermented yogurt in the 96 low-well plates in concentration of 50 CFU/g in each well. The plates were then incubated at 7±1 °C, with a scanner scanning the surface of the plates, for 52 days. The resulting color changes on the surface was used to indicate yeast inhibition. In FIG. 3, the time to outgrowth of the yeast is estimated from the color values and shows the inhibition of D. hansenii.

The results presented in FIG. 3 show that L. rhamnosus DSM 34195 fully inhibits D. hansenii at all examined dosages, whereas DSM 33515 shows low inhibition at 5×10⁶ CFU/g. The inhibition increases with the dosage, reaching complete inhibition of D. hansenii at a dosage of 2×10⁷ CFU/g.

### Example 3 Antifungal Effect of L. rhamnosus DSM 34195 Compared with L. rhamnosus DSM 33515

For the demonstration of the inhibitory effect of L. rhamnosus DSM 34195 against mold, a semi-quantitative agar-assay was used. For the inhibitory effect against yeast, a challenge test following the growth (Log (CFU/g)) of different target contaminants in a fermented milk sample (yogurt) was used.

A homogenized milk base consisting of 2.8% protein, 1.2% fat and 10% sucrose was heat-treated at 95±1 °C for 5 min and cooled immediately. A commercial starter culture (F-DVS YF-L812) was used to inoculate at 500 U per 2500 L, and the inoculated milk was distributed into 200 ml bottles. In selected bottles, L. rhamnosus DSM 34195 was used to inoculate in total concentrations of 5×10⁶ CFU/g, 1×10⁷ CFU/g, 2×10⁷ CFU/g and 3×10⁷ CFU/g, respectively. In other bottles, L. rhamnosus DSM 33515 was used to inoculate in total concentration of 3×10⁷ CFU/g. Bottles only inoculated with the starter culture were used as reference. All bottles were incubated in a water bath at 43±1 °C and fermented at these conditions until pH of 4.55+0.1 was reached. After fermentation, the bottles were vigorously stirred to break the coagulum and immediately cooled in a cooling chamber.

For the yeast challenge, fermented milk samples were inoculated with 50 cfu/g the target contaminant D. hansenii CHCC16374. The inoculated samples were stored at 7 °C for 24 days and five times during storage the yeast growth was evaluated as total yeast count.

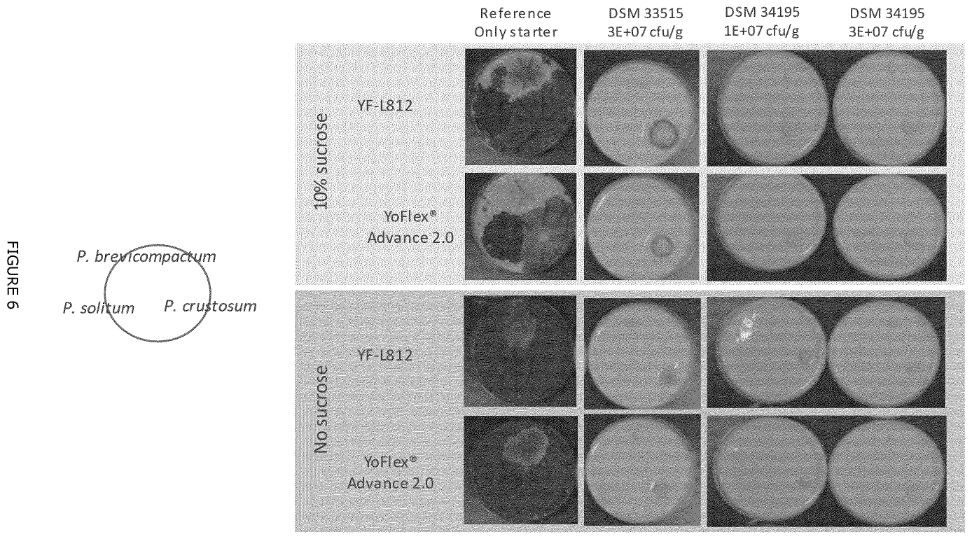

For the mold challenge, the 200 ml fermented milk was warmed to a temperature of 40 °C and 40 ml of a 5% sterile agar solution that had been melted and cooled down to 60 °C. This solution of fermented milk and agar was then poured into sterile Petri dishes and the plates were dried in a LAF bench for 30 min. Spore suspension of the following six different molds were spotted in concentration of 500 spores/spot onto the agar plates (3 molds per plate as indicated in FIG. 5): P. brevicompactum DSM 32094, P. crustosum DSM 33517, P. solitum DSM 32093, P. carneum DSM 33520, P. paneum DSM 33519 and P. roqueforti DSM 33518. Three molds were spotted on each plate and the target contaminants were added in concentrations of 500 spores/spot. Plates were incubated at 22±1 °C for 8 days and regularly examined for mold growth.

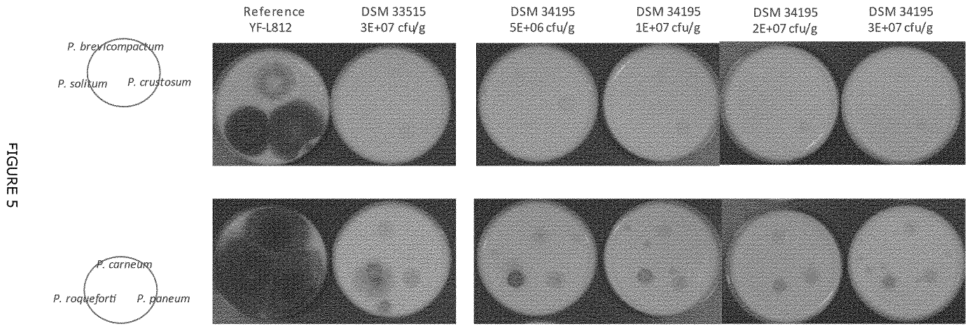

The effect at low inoculation rate was again confirmed in the yeast challenge test from which the growth curves (Log (CFU/g)) are presented in FIG. 4. Growth inhibition of D. hansenii was observed when inoculated in a fermented product with starter culture and L. rhamnosus DSM 33515 dosed at 3×10⁷ CFU/g. Surprisingly, when inoculated with L. rhamnosus DSM 34195, even higher inhibition can be seen at all inoculation levels tested—5×10⁶ CFU/g, 1×10⁷ CFU/g, 2×10⁷ CFU/g and 3×10⁷ CFU/g.

Results of the agar-assay are presented in FIG. 5, showing that all of the tested molds grew very well on the agar plates made from milk fermented only with the starter culture (reference). However, when L. rhamnosus DSM 34195 was present during milk fermentation the resulting plates inhibited growth of the six Penicillium species tested, and inhibition was clearly observed at all dose levels from 5×10⁶ CFU/g to 3×107 CFU/g. Surprisingly, the effect of L. rhamnosus DSM 34195 dosed at lowest rate of 5×10⁶ CFU/g was at levels similar to L. rhamnosus DSM 33515 dosed at 3×10⁷ CFU/g.

Conclusion: The result shows that DSM 34195 has higher anti-yeast and anti-mold effect than DSM 33515.

### Example 4 Antifungal Effect of L. rhamnosus DSM 34195 in Combination with Different Starter Cultures in Different Milk Bases

4.1 Combination with Different Starter Cultures and Different Milk Bases

For the demonstration of the inhibitory effect of L. rhamnosus bacteria DSM 34195 against mold, a semi-quantitative agar-assay was used. For the inhibitory effect against yeast, a challenge test following the growth (Log (cfu/g)) of different target contaminants in a fermented milk (yogurt) was used.

Two milk bases consisting of 2.8% protein, 1.2% fat, with or without 10% sucrose, was homogenized and heat-treated at 95±1 °C for 5 min and cooled immediately. Commercial starter culture containing Lactobacillus delbrueckii subsp. bulgaricus and Streptococcus thermophilus (F-DVS YF-L812 or YoFlex Advance 2.0 from Chr. Hansen A/S, Denmark) was used to inoculate at 500 U per 2500 L, and the inoculated milk was distributed into 200 ml bottles. In selected bottles, L. rhamnosus DSM 34195 was used to inoculate in total concentrations of 1×10⁷ CFU/g and 3×10⁷ CFU/g. In other bottles, L. rhamnosus DSM 33515 was used to inoculate in total concentration of 3×10⁷ CFU/g. Bottles only inoculated with the starter culture were used as reference. All bottles were incubated in a water bath at 43±1 °C and fermented at these conditions until pH of 4.55±0.1 was reached. After fermentation, the bottles were vigorously stirred to break the coagulum and immediately cooled in a cooling chamber.

For the mold challenge, the 200 ml fermented milk was warmed to a temperature of 40 °C and added with 40 ml of a 5% sterile agar solution that had been melted and cooled down to 60 °C. This solution of fermented milk and agar was then poured into sterile Petri dishes and the plates were dried in a LAF bench for 30 min. Spore suspension of the following three different molds were spotted in concentration of 500 spores/spot onto the agar plates: P. brevicompactum DSM 32094, P. crustosum DSM 33517, P. solitum DSM 32093. The three molds were spotted on one plate and the target contaminants were added in concentrations of 500 spores/spot. Plates were incubated at 22±1 °C for 11 days and regularly examined for mold growth.

For the yeast challenge, fermented milk samples were inoculated with 50 cfu/g the target contaminant D. hansenii CHCC16374. The inoculated samples were stored at 7 °C for 27 days and six times during storage the yeast growth was evaluated as total yeast count.

Results of the agar-assay are presented in FIG. 6, showing that all the tested molds grew very well on the agar plates made from milk fermented only with starter cultures and in both milk bases (Reference). However, when L. rhamnosus DSM 34195 was present during milk fermentation, the resulting plates inhibited growth of the three Penicillium species tested, and inhibition was clearly observed for both starter cultures, milk bases and all dose levels. The inhibitory effect of L. rhamnosus DSM 34195 was similar or higher than L. rhamnosus DSM 33515 when both were dosed at 3×10⁷ CFU/g.

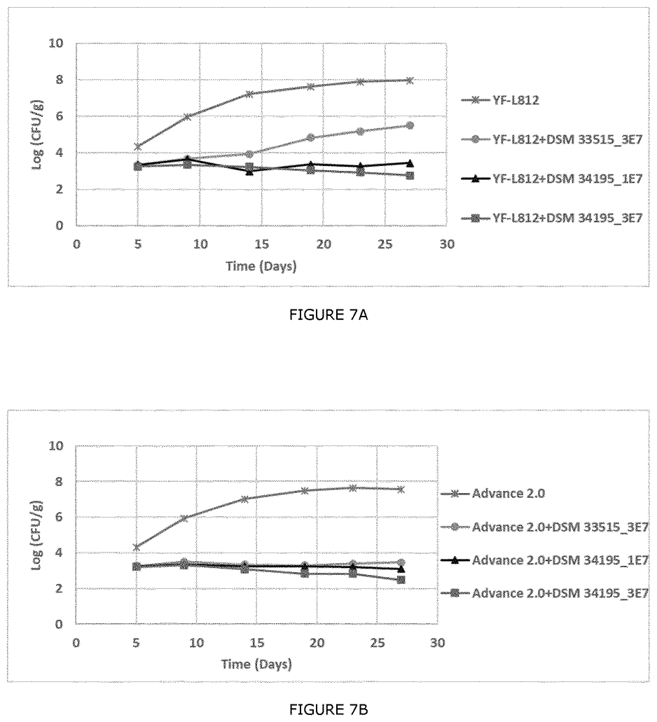

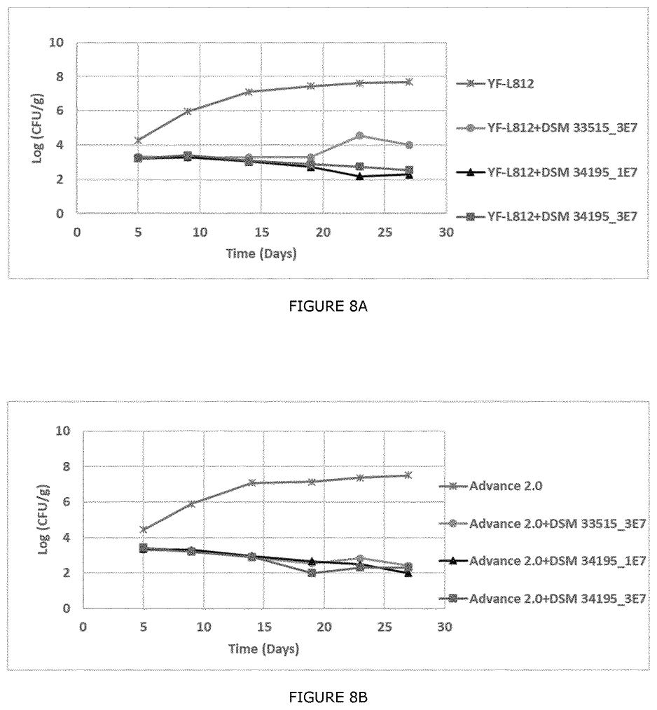

FIGS. 7 and 8 show the growth curves for D. hansenii in fermented products with and without 10% sugar using two starter cultures YF-L812 or YoFlex Advance 2.0 and L. rhamnosus DSM 33515 dosed at 3×10⁷ CFU/g or L. rhamnosus DSM 34195 dosed at 1×10⁷ CFU/g and 3×10⁷ CFU/g. L. rhamnosus DSM 34195 shows consistent and high inhibition of D. hansenii at both inoculation levels in combination with the two starter cultures and in the two milk bases. Less consistent inhibition is observed with L. rhamnosus DSM 33515 especially in the fermented product 10% sucrose and with YF-L812 as starter culture (7A).

4.2 Combination with Different Starter Cultures

Improved anti-mold and anti-yeast effect was also observed in a trial producing fermented milk products with different starter culture. For this, a milk base which consists of 2.8% protein, 1.2% fat and 10% sucrose was used. The milk base was prepared by homogenized and heat-treated at 95±1 °C for 5 min, filled in 3-liter buckets and cooled immediately. At start of fermentation, the buckets with milk were inoculated with commercial starter culture containing Lactobacillus delbrueckii subsp. bulgaricus and Streptococcus thermophilus (F-DVS YoFlex Mild 1.0 or YoFlex Premium 1.0 from Chr. Hansen A/S, Denmark) at 500 U per 2500 L. L. rhamnosus DSM 34195 or L. rhamnosus DSM 33515 was used to inoculate in total concentrations of 1×10⁷ CFU/g and 3×10⁷ CFU/g, respectively. Two buckets were used as a reference and was only inoculated with one of the starter cultures. All buckets were incubated in a water bath at 43±1 °C and fermented at these conditions until pH of 4.60±0.1 was reached. After fermentation, the buckets were vigorously stirred to break the coagulum and cooled to 25±1 °C, dispensed into 250 ml cups and immediately placed at cold storage at 5-7 °C.

For the mold challenge, the 200 ml fermented milk was warmed to a temperature of 40 °C and added with 40 ml of a 5% sterile agar solution that had been melted and cooled down to 60 °C. This solution of fermented milk and agar was then poured into sterile Petri dishes and the plates were dried in a LAF bench for 30 min. Spore suspension of the following three different molds were spotted in concentration of 500 spores/spot onto the agar plates: P. carneum DSM 33520, P. paneum DSM 33519 and P. roqueforti DSM 33518. The three molds were spotted on one plate and the target contaminants were added in concentrations of 500 spores/spot. Plates were incubated at 7±1 °C for 34 days and regularly examined for mold growth.

For the yeast challenge, fermented milk samples were inoculated with 50 cfu/g the target contaminants Debaryomyces hansenii CHCC16374 or Torulaspora delbrueckii

CHCC17090 (Chr. Hansen Culture Collection). The inoculated samples were stored at 7 °C for 28 days and five times during storage the yeast growth was evaluated as total yeast count.

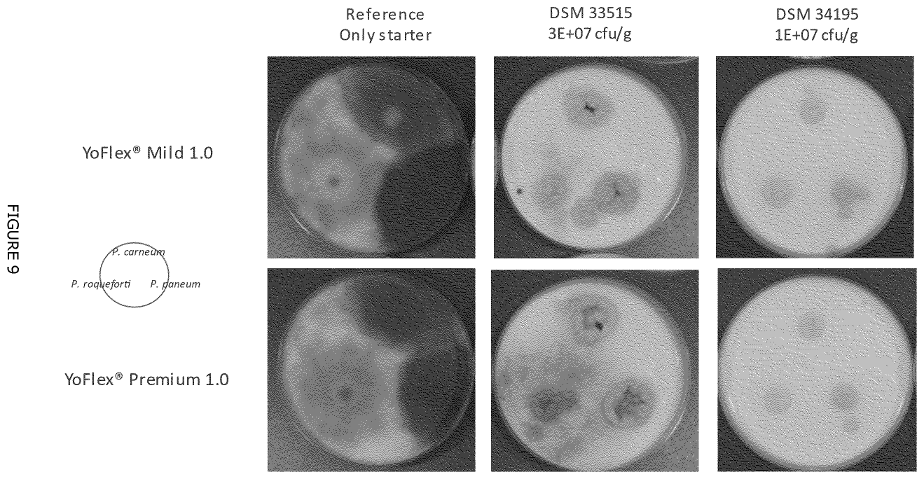

Results of the agar-assay are presented in FIG. 9, showing that all the tested molds grew very well on the agar plates made from milk fermented only with starter cultures (Reference). However, when L. rhamnosus DSM 34195 was present during milk fermentation, the resulting plates inhibited growth of the three Penicillium species tested, and inhibition was clearly observed. The effect of L. rhamnosus DSM 34195 inoculated at 1×10⁷ CFU/g was even higher than L. rhamnosus DSM 33515 inoculated at 3×10⁷ CFU/g.

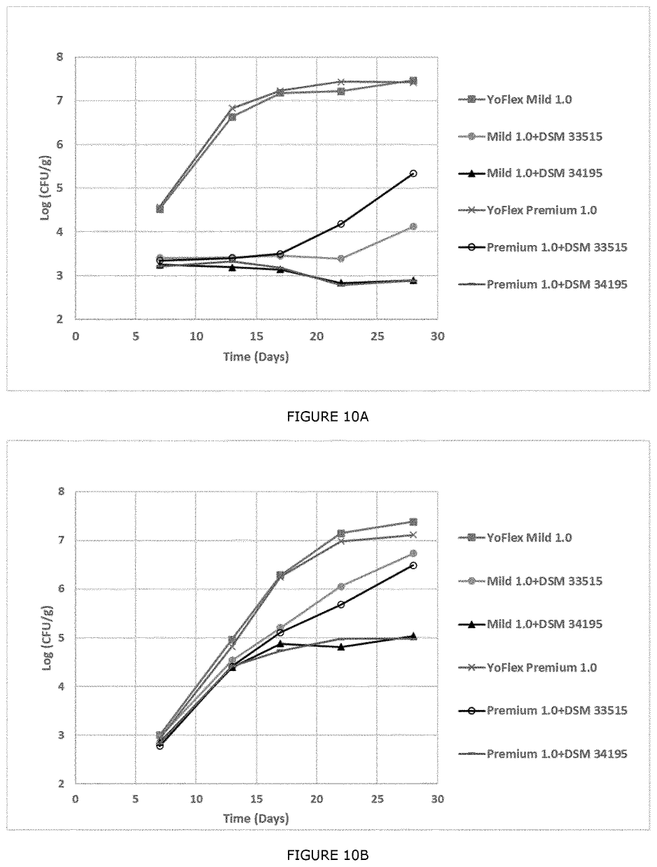

FIG. 10 shows the growth curves for D. hansenii (10A) and T. delbreuckii (10B) during 28 days of storage at 7 °C. The growth of the two target contaminants was inhibited to the highest degree by of L. rhamnosus DSM 34195 compared to L. rhamnosus DSM 33515, even at an inoculation level at 1×10⁷ CFU/g compared to 3×10⁷ CFU/g which was used for L. rhamnosus DSM 33515. Higher inhibition can be observed in both cases using the starter culture F-DVS YoFlex Mild 1.0 or YoFlex Premium 1.0.

Conclusion: L. rhamnosus DSM 34195 shows higher and more consistent anti-yeast and anti-mold effect than L. rhamnosus DSM 33515 in combination with different starter cultures and in different milk bases.

### Example 5 Low Post-Acidification of L. rhamnosus DSM 34195 in Fermented Milk Product

This example demonstrates the impact of post-acidification of L. rhamnosus DSM 34195 compared to L. rhamnosus DSM 33515. For this, a homogenized milk base consisting of 2.8% protein, 1.2% fat and 10% sucrose was heat-treated at 95±1 °C for 5 min and cooled immediately. Commercial starter culture (F-DVS YF-L812 or F-DVS YoFlex Advance 2.0) was used to inoculate at 500 U per 2500 L, and the inoculated milk was distributed into 3-liter buckets. One bucket was inoculated with L. rhamnosus DSM 33515 in total concentration of 3×10⁷ CFU/g, one bucket was inoculated with the L. rhamnosus DSM 34195 in total concentration of 1×10⁷ CFU/g, and one bucket was used as a reference and was only inoculated with the starter culture. All buckets were incubated in a water bath at 43±1 °C and fermented at these conditions until pH of 4.60±0.1 was reached. After fermentation, the buckets were vigorously stirred to break the coagulum, cooled in cold water to 25±1 °C, dispensed into 200 ml bottles and immediately placed at cold storage at 5-7 °C.

To monitor the effect on post acidification, the 2x three fermented milk samples (starter-only, starter+DSM 34915 and starter+DSM 33515) were stored at 25±1 °C for 28 days and pH was measured on day 1, 7, 14, 21 and 28.

FIG. 11 shows the effect on post-acidification when combined with F-DVS YF-L812 (11A) or F-DVS YoFlex Advance 2.0 (11B). The addition of L. rhamnosus DSM 33515 induced more post-acidification than L. rhamnosus DSM 34195.

### Example 6 Antifungal Effect and Low Post-Acidfication of L. rhamnosus DSM 34195 Compared with L. rhamnosus DSM 33515 in Crema Ácida

For the demonstration of the inhibitory effect of L. rhamnosus DSM 34195 against mold, a semi-quantitative agar-assay was used. For the inhibitory effect against yeast, a challenge test following the growth (Log (CFU/g)) of different target contaminants in a fermented milk sample (crema ácida) was used.

A homogenized milk base consisting of 2.8% protein, 14-31% fat and 4% carbohydrates was heat-treated at 85±1 °C for 15 sec, followed by homogenization and immediate cooling. The crema ácida was then chemically acidified with a citric acid solution to adjust the pH to 4.6 and distributed into 200 ml bottles. In selected bottles, L. rhamnosus DSM 34195 was used to inoculate a total concentration of 1×10⁷ CFU/g. In other bottles, L. rhamnosus DSM 33515 was used to inoculate a total concentration of 3×10⁷ CFU/g. Bottles without addition of L. rhamnosus were used as a reference. All bottles were stored after inoculation at 7 °C.

For the yeast challenge, fermented milk samples were inoculated with 50 cfu/g the target contaminant D. hansenii CHCC16374. The inoculated samples were stored at 7 °C for 24 days and five times during storage the yeast growth was evaluated as total yeast count.

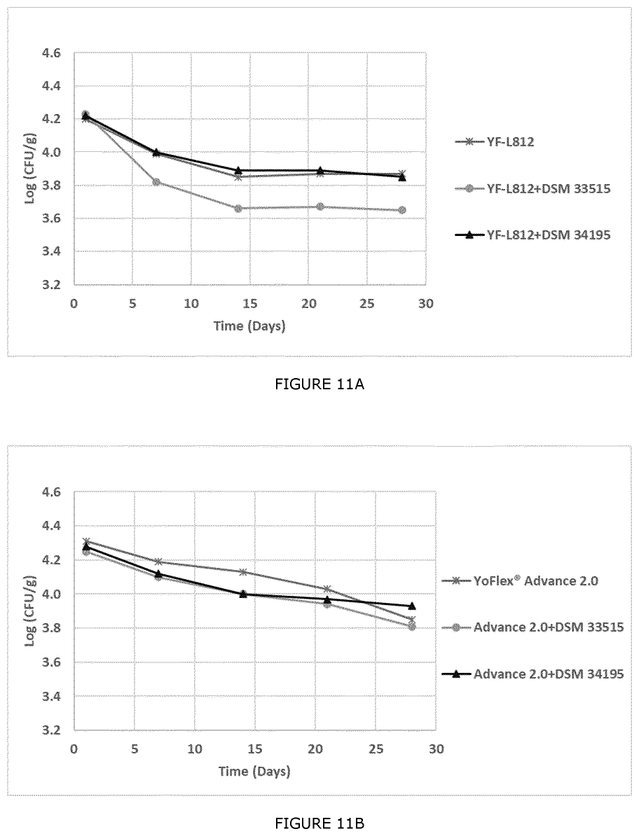

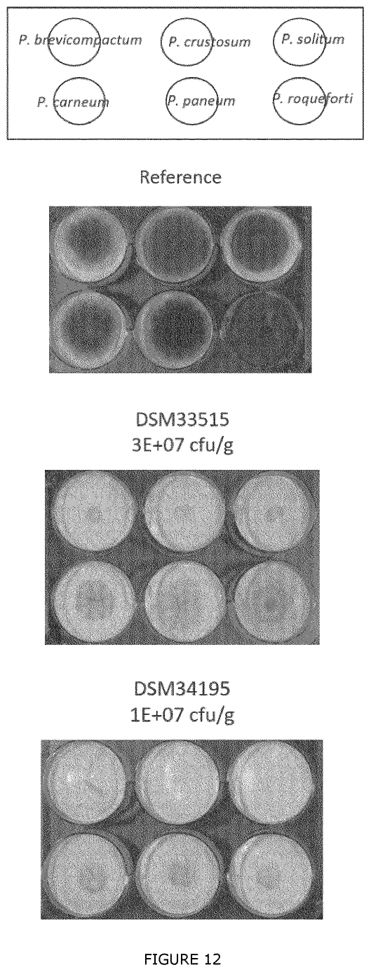

For the mold challenge, the 200 ml fermented milk was warmed to a temperature of 40 °C and 40 ml of a 5% sterile agar solution that had been melted and cooled down to 60 °C. This solution of fermented milk and agar was then poured into sterile 6-well plates and the plates were dried in a LAF bench for 30 min. Spore suspension of the following six different molds were spotted in concentration of 500 spores/spot onto the plates (One mold per well as indicated in FIG. 12): P. brevicompactum DSM 32094, P. crustosum DSM 33517, P. solitum DSM 32093, P. carneum DSM 33520, P. paneum DSM 33519 and P. roqueforti DSM 33518 and plates were incubated 12±1 °C for 15 days and 7±1 °C for 22 days regularly examined for mold growth. Plates incubated at 22±1 °C for 7 days, were inoculated with two molds, including P. crustosum DSM 33517 and P. roqueforti DSM 33518.

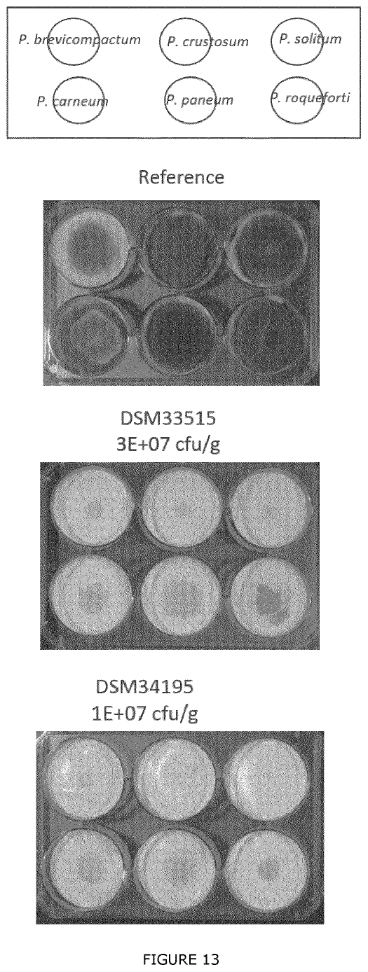

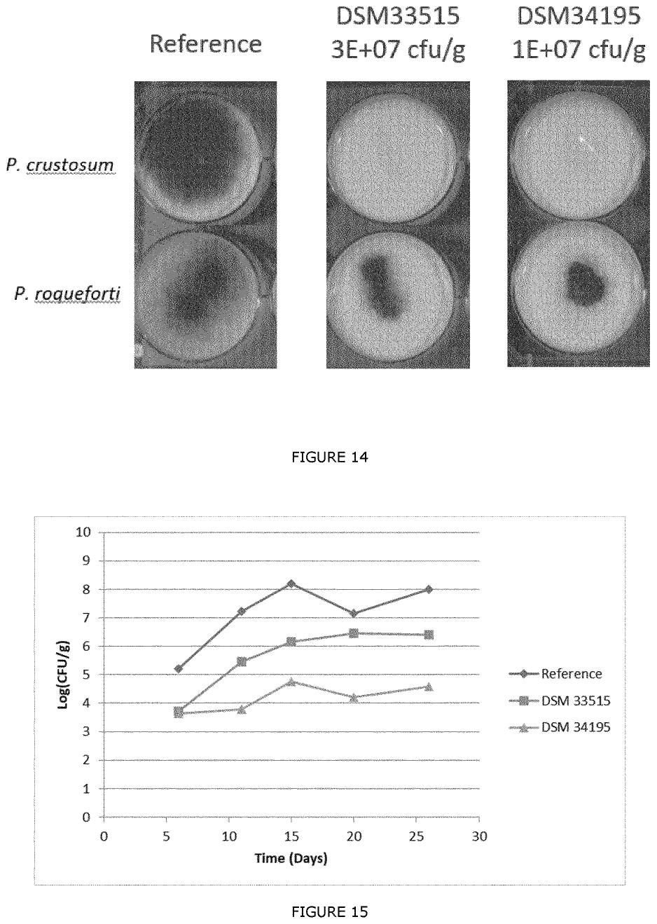

Results of the agar-assay are presented in FIG. 12-14, showing that all of the tested molds grew very well on the agar plates made from crema acida without the addition of L. rhamnosus (reference). However, when L. rhamnosus DSM 34195 was added, the resulting plates showed growth inhibition of the six Penicillium species tested when incubated at 7 °C (FIG. 12) or 12 °C (FIG. 13) and for the two molds when incubated at 22 °C (FIG. 14).

The antifungal activity was confirmed in the yeast challenge test, growth curves (Log (CFU/g)) are presented in FIG. 15. Growth inhibition of D. hansenii was observed when inoculated in the fermented product with L. rhamnosus DSM 33515 dosed at 3×10⁷ CFU/g but even higher levels of inhibition were observed when inoculated with L. rhamnosus DSM 34195 at 1×10⁷ CFU/g.

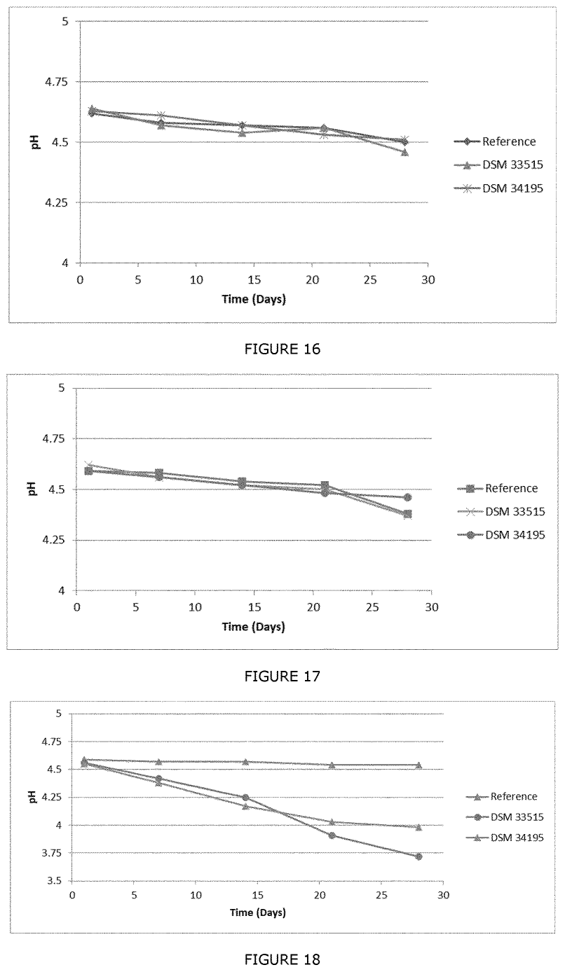

Post-acidification was monitored by measuring pH for samples stored at 7 °C, 12 °C and 25 °C for 28 days, pH was measured on days 1, 7, 14, 21, and 28. FIGS. 14A-C show lower post-acidification for L. rhamnosus DSM 34195 than L. rhamnosus DSM 33515 at the end of shelf-life at 7 °C (FIG. 16), 12 °C (FIG. 17) and 25 °C (FIG. 18).

Conclusion: The result shows that DSM 34195 has higher anti-yeast and anti-mold effect and lower post-acidification than DSM 33515 in crema ácida, even when DSM 34195 is applied at lower dosage.

1. Bacterium of the species Lacticaseibacillus rhamnosus deposited as DSM 34195 or a mutant thereof, wherein the mutant thereof: is obtained by using as starting material DSM 34195, is different from DSM 33515 and DSM 23035, has retained or improved properties compared to DSM 34195, has improved properties compared to DSM 33515, wherein the properties are antifungal effect or activity and reduced post-acidification.

2. A composition comprising the bacterium of the species Lacticaseibacillus rhamnosus deposited as DSM 34195 or a mutant thereof according to claim 1.

3. The composition according to claim 2, wherein the composition further comprises cryoprotectants, lyoprotectants, antioxidants, nutrients, fillers, flavorants or mixtures thereof; preferably wherein nutrients are sugars, amino acids, fatty acids, minerals, trace elements, vitamins; preferably wherein fillers are preferably lactose, maltodextrin.

4. The composition according to claim 2, wherein the bacterium or a mutant thereof is in a concentration of at least 109 colony forming units (CFU)/g, or in a concentration of at least 10¹⁰ CFU/g, or in a concentration of at least 1011 CFU/g.

5. The composition according to claim 2, wherein the composition is frozen or freeze-dried.

6. The composition according to claim 2, wherein the composition further comprises a starter culture.

7. The composition according to claim 6, wherein the starter culture comprises Streptococcus thermophilus and Lactobacillus delbrueckii subsp. bulgaricus.

8. A method of producing a fermented milk product, comprising adding the bacterium of the species Lacticaseibacillus rhamnosus or a mutant thereof according to claim 1 and fermenting the mixture at a temperature between about 22 °C and about 43 °C until a pH of 4.6 or less than 4.6 is reached.

9. A fermented milk product comprising the bacterium of the species Lacticaseibacillus rhamnosus deposited as DSM 34195 or a mutant thereof according to claim 1.

10. The fermented milk product according to claim 9, wherein the product is cheese, yoghurt, crema ácida, or sour cream.

11. A fermented milk product to obtained by the method according to claim 8.

12. The fermented milk product according to claim 9, wherein the bacterium of the species Lacticaseibacillus rhamnosus or a mutant thereof is present in a concentration of at least 10⁷ CFU/g.

13. The fermented milk product according to claim 9, wherein the product is stored at a temperature above 7 °C.

14. A feed, feed or pharmaceutical product comprising of the species Lacticaseibacillus rhamnosus deposited as DSM 34195 or a mutant thereof according to claim 1.

15. A food, feed or pharmaceutical product obtained by the method of claim 8.
